(19) United States
(12) Patent Application Publication
(10) Pub. No.: US 2007/0099959 A1
(43) Pub. Date: May 3, 2007

(54) O-Aminophenol derivatives and colorants containing these compounds

(75) Inventors: Cecile Pasquier, Marly (CH); Nadia Duc-Reichlin, Lully FR (CH); Hans-Jurgen Braun, Ueberstorf (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/524,149

(22) Filed: Sep. 20, 2006

Related U.S. Application Data
(63) Continuation of application No. PCT/EP05/06845, filed on Jun. 24, 2005.

(30) Foreign Application Priority Data
Aug. 25, 2004 (DE) 102004041137.9

(51) Int. Cl. C07D 211/06; A61K 31/445; A61K 31/44; A61K 31/165; A61K 31/34; C07C 215/28

(52) U.S. Cl. 514/317; 514/352; 514/471; 514/619; 546/226; 546/308; 549/480; 564/196

(57) ABSTRACT

The object of the present invention is novel o-aminophenol derivatives of Formula (I), or physiologically compatible water-soluble salts thereof, 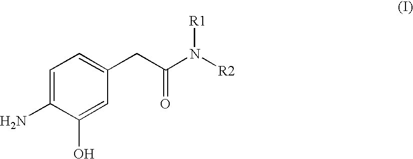 as well as an agent for the coloring of keratin fibers, especially of hair, which contains at least one o-aminophenol derivative of Formula (I).

### FIELD OF THE INVENTION

The present invention relates to new o-aminophenol derivatives substituted in the 5-position as well as agents that contain these compounds for the coloring of keratin fibers, especially human hair.

### BACKGROUND OF THE INVENTION

In the area of coloring of keratin fibers, especially the coloring of hair, oxidative dyes have acquired a substantial significance. Coloring takes place through the reaction of certain developer substances with certain coupler substances in the presence of a suitable oxidizing agent. Particular examples of developer substances that can be used include 2,5-diaminotoluene, 2,5-diaminophenylethyl alcohol, p-aminophenol, 1,4-diaminobenzene, and 4,5-diamino-1-(2-hydroxyethyl)pyrazole, while examples of coupler substances that can be named include resorcinol, 2-methylresorcinol, 1-naphthol, 3-aminophenol, m-phenylenediamine, 2-amino-4-(2′-hydroxyethyl)aminoanisole, 1,3-diamino-4-(2′-hydroxyethoxy)benzene, and 2,4-diamino-5-fluorotoluene.

Regarding the oxidative dyes that are used in the coloring of human hair, there are numerous requirements imposed, in addition to coloring to a desired intensity. Thus, the dye must be safe from a toxicological and dermatological perspective, and the hair coloring obtained must exhibit good lightfastness, permanent wave fastness, acid fastness, and resistance to rubbing. In any case, such coloring must remain stable to the influence of light, rubbing, and chemical agents over a period of at least 4-6 weeks. An additional requirement is that it should be possible to produce a broad palette of different color shades by combining suitable developer substances and coupler substances.

A particular problem is posed by the shading of light color tones with respect to balancing the dye uptake from the hairline to the hair tip and with respect to the durability of the shades during a permanent wave treatment. The use of direct-penetrating, yellow-colored aromatic nitro dyes together with oxidative hair colorant precursors can solve the problem described to some extent; however, the stability of the coloring over a period of several weeks is often unsatisfactory.

A solution to this problem can be found in DE-OS 28 33 989 in which the use of 2-amino-5-methylphenol as an oxidative yellow-colored dye in oxidative hair colorants was suggested. This compound is well suited to the production of light blonde tones and golden tones, but it does not completely meet the established requirements, mainly with respect to the resistance of the hair coloring to the effects of permanent wave agents.

Surprisingly, it has now been found that o-aminophenol derivatives of the general Formula (I) fulfill the established requirements to a particularly great extent. Thus, when these substances are used with most of the known coupler or developer substances, intense color shades are obtained in which the colors are exceptionally resistant to washing and stable when subjected to permanent waves.

The object of the present invention is thus novel o-aminophenol derivatives of Formula (I), or physiologically compatible water-soluble salts thereof, 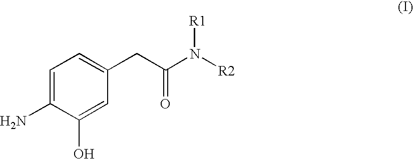
wherein
R1 and R2, independently from one another, can represent either hydrogen, a saturated or unsaturated (C₁-C₆) alkyl group, a (C₁-C₆) hydroxyalkyl group, a (C₂-C₆) dihydroxyalkyl group, a (C₁-C₃)-alkoxy-( C₁-C₃)-alkyl group, a (C₁-C₃)-hydroxyalkyl-( C₁-C₃)-alkoxy group, a (C₁-C₆) aminoalkyl group, a (C₁-C₄)-alkylamino-(C₁-C₄)-alkyl group, a di-( C₁-C₄)-alkylamino-(C₁-C₄)-alkyl group, a (C₁-C₆) acetylaminoalkyl group, a (C₁-C₆) cyanoalkyl group, a (C₁-C₆) carboxyalkyl group, a (C₁-C₆) aminocarbonylalkyl group, an unsubstituted or substituted phenyl group, a benzyl group, a furfuryl group, a tetrahydrofurfuryl group or a pyridylmethyl group, or R1 and R2 together with the N-atom can, if necessary, form a substituted, saturated, or unsaturated 4-membered to 8-membered ring, which can contain an additional heteroatom-especially an O-, S-, or N-atom.

Examples of suitable compounds of Formula (I) that can be named include the following compounds:

1-[(4-amino-3-hydroxyphenyl)acetyl]pyrrolidine, 1-[(4-amino-3-hydroxyphenyl)acetyl]piperidine, 1-[(4-amino-3-hydroxyphenyl)acetyl]-4-hydroxypiperidine, 1-[(4-amino-3-hydroxyphenyl)acetyl]morpholine, 1-[(4-amino-3-hydroxyphenyl)acetyl]piperazine, 1-[(4-amino-3-hydroxyphenyl)acetyl]-4-methylpiperazine, 2-(4-amino-3-hydroxyphenyl)acetamide, 2-(4-amino-3-hydroxyphenyl)-N-methylacetamide, 2-(4-amino-3-hydroxyphenyl)-N-ethylacetamide, 2-(4-amino-3-hydroxyphenyl)-N-propylacetamide, 2-(4-amino-3-hydroxyphenyl)-N-butylacetamide, 2-(4-amino-3-hydroxyphenyl)-N-(2-hydroxyethyl)acetamide, 2-(4-amino-3-hydroxyphenyl)-N-(3-hydroxypropyl)acetamide, 2-(4-amino-3-hydroxyphenyl)-N-(4-hydroxybutyl)acetamide, 2-(4-amino-3-hydroxyphenyl)-N-(2,3-dihydroxypropyl)acetamide, 2-(4-amino-3-hydroxyphenyl)-N-[2-(methyloxy)ethyl]acetamide, 2-(4-amino-3-hydroxyphenyl)-N-[3-(methyloxy)propyl]acetamide, 2-(4-amino-3-hydroxyphenyl)-N-{2-[(2-hydroxyethyl)oxy]ethyl}acetamide, 2-(4-amino-3-hydroxyphenyl)-N-(cyanomethyl)acetamide, 2-(4-amino-3-hydroxyphenyl)-N-(2-aminoethyl)acetamide, 2-(4-amino-3-hydroxyphenyl)-N-(3-aminopropyl)acetamide, 2-(4-amino-3-hydroxyphenyl)-N,N-dimethylacetamide, 2-(4-amino-3-hydroxyphenyl)-N,N-diethylacetamide, 2-(4-amino-3-hydroxyphenyl)-N,N-dipropylacetamide, 2-(4-amino-3-hydroxyphenyl)-N,N-diisopropylacetamide, 2-(4-amino-3-hydroxyphenyl)-N,N-dibutylacetamide, 2-(4-amino-3-hydroxyphenyl)-N,N-bis(2-hydroxyethyl)acetamide, 2-(4-amino-3-hydroxyphenyl)-N,N-bis(3-hydroxypropyl)acetamide, 2-(4-amino-3-hydroxyphenyl)-N-(tetrahydro-2-furanylmethyl)acetamide, 2-(4-amino-3-hydroxyphenyl)-N-(2-furanylmethyl)acetamide, 2-(4-amino-3-hydroxyphenyl)-N-phenylacetamide, 2-(4-amino-3-hydroxyphenyl)-N-(4-hydroxyphenyl)acetamide, 2-(4-amino-3-hydroxyphenyl)-N-(3-hydroxyphenyl)acetamide, 2-(4-amino-4-hydroxyphenyl)-N-(4-methoxyphenyl)acetamide, 2-(4-amino-4-hydroxyphenyl)-N-(3-methoxyphenyl)acetamide, 2-(4-amino-4-hydroxyphenyl)-N-(2,4-dimethoxyphenyl)acetamide, 2-(4-amino4-hydroxyphenyl)-N-(2-pyridinyl)acetamide, 2-(4-amino-4-hydroxyphenyl)-N-(3-pyridinyl)acetamide, as well as physiologically compatible water-soluble salts thereof.

Preferred compounds of Formula (I) are those in which:

(i) R1 and R2 are either hydrogen or a substituted or unsubstituted (C₁-C₆) alkyl group; or,

(ii) R1 and R2 form, if necessary, a substituted, saturated 4-membered to 8-membered ring, which can contain an O-, S-, or N-atom.

The following compounds of Formula (I) can be named in particular:

1-[(4-amino-3-hydroxyphenyl)acetyl]pyrrolidine, 1-[(4-amino-3-hydroxyphenyl)acetyl]piperidine, 2-(4-amino-3-hydroxyphenyl)-N-propylacetamide, 2-(4-amino-3-hydroxyphenyl)-N,N-diethylacetamide, as well as physiologically compatible water-soluble salts thereof.

The compounds of Formula (I) can be used either as the free bases or in the form of their physiologically compatible salts with inorganic or organic acids such as, for example, hydrochloric acid, sulfuric acid, phosphoric acid, acetic acid, propionic acid, lactic acid, or citric acid.

The preparation of the 2-aminophenol derivatives of the present invention of Formula (I) can be achieved through the use of known synthesis methods, for example, according to Scheme 1 below through a carbon homologation of a protected or unprotected 3-hydroxy4-nitrobenzaldehyde, via its dibromalkene derivative, analogous to the synthesis pathway described in Tetrahedron 58 (2002), page 9925-9932, followed by reduction and cleavage of the protecting groups required for the reaction if necessary, whereby R represents hydrogen or a protecting group, as described, for example, in the book Protective Groups in Organic Synthesis, Chapter 3, Wiley Interscience, 1991.

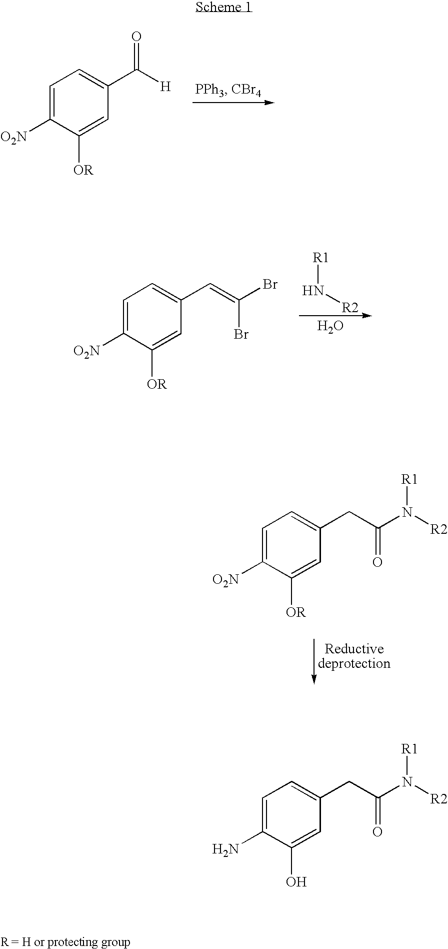

The o-aminophenol derivatives of Formula (I) have good water solubility and enable coloring with excellent color intensity and color fastness, especially with respect to color fastness when subjected to washing and rubbing.

An additional object of the present invention is thus an agent for the coloring of keratin fibers such as, for example, wool, furs, feathers, or hair, and especially human hair, based on a developer-substance-coupler-substance combination, wherein the agent contains at least one o-aminophenol derivative of Formula (I) or the physiologically compatible water-soluble salts thereof.

The colorant of the present invention contains the o-aminophenol derivatives of Formula (I) in a total amount of from approximately 0.005 to 10 percent by weight, where a quantity of from approximately 0.01 to 5 percent by weight is preferred, and of from 0.01 to 2.5 percent by weight is especially preferred.

The o-aminophenol derivatives of Formula (I) will color keratinous material with yellow tones without the addition of other dyes.

To realize additional shades of color, conventional oxidative dyes can be added, for example, developer substances or coupler substances, singly or in combination with each other.

Examples of preferred developer substances that can be considered include 1,4-diaminobenzene

(p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 4-(2,5-diaminophenyl)-2-((diethylamino)methyl)thiophene, 2-Chlor-3-(2,5-diaminophenyl)thiophene, 1,4-diamino-2-(pyridin-3-yl)benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-(2-(acetylamino)ethoxy)-1,4-diaminobenzene, 4-phenylaminoaniline, 4-dimethylaminoaniline, 4-diethylaminoaniline, 4-dipropylaminoaniline, 4-[ethyl-(2-hydroxyethyl)amino]aniline, 4-[di-(2-hydroxyethyl)amino]aniline, 4-[di-(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(1-hydroxyethyl)benzene, 1,4-diamino-2-(2-hydroxyethyl)benzene, 1,4-diamino-2-(1-methylethyl)benzene, 1,3-bis-[(4-aminophenyl)(2-hydroxyethyl)amino]-2-propanol, 1,4-bis-[(4-amino-phenyl)amino]butane, 1,8-bis-(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1l)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol, 2-amino-5-methylphenol and 1,2,4-trihydroxybenzene.

Examples of preferred coupler substances that can be considered include N-(3-dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di-[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1,3-diamino4-(2,3-dihydroxypropoxy)benzene, 1,3-diamino4-(3-hydroxypropoxy)benzene, 1,3-diamino4-(2-methoxyethoxy)benzene, 2,4-diamino-1,5-di-(2-hydroxyethoxy)benzene, 1-(2-aminoethoxy)-2,4-diaminobenzene, 2-amino-1-(2-hydroxyethoxy)4-methylaminobenzene, 2,4-diaminophenoxyacetic acid, 3-[di-(2-hydroxyethyl)amino]aniline, 4-amino-2-di-[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di-(2,4-diaminophenoxy)propane, di-(2,4-diaminophenoxy)methane, 1,3-diamino-2,4-dimethoxybenzene, 2,6-bis-(2-hydroxyethyl)aminotoluene, 4-hydroxyindole, 3-dimethylaminophenol, 3-diethylaminophenol, 5-am ino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]phenol, 3-[(2-methoxyethylamino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 2,6-dihydroxy-3,4-dimethylpyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthyl acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylendioxyphenol, 3,4-methylendioxyaniline, 5-[(2-hydroxyethyl)amino]-1,3-benzodioxole, 6-bromo-1-hydroxy-3,4-methylendioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole, and 2,3-indolinedione.

The above-named developer substances and coupler substances can be used in the colorant of the present invention either singly or in mixtures with each other, where the total amount of developer substances and coupler substances in the agent of the present invention is from approximately 0.01 to 12 percent by weight, especially from approximately 0.2 to 6 percent by weight.

Moreover, the colorant of the present invention can also contain additional color components, for example, 4-(2,5-diaminobenzylamino)aniline or 3-(2,5-diaminobenzylamino)aniline, as well as conventional direct-penetrating dyes, for example, triphenylmethane dyes such as 4-[(4′-aminophenyl)-(4′-imino-2″,5″-cyclohexadien-1″-ylidene)methyl]-2-methylaminobenzene monohydrochloride (C.I. 42 510) and 4-[(4′-amino-3′-methylphenyl)-(4″-imino-3″-methyl-2″,5″cyclohexadien-1″-ylidene]methyl)-2-methylaminobenzene monohydrochloride (C.I. 42 520), aromatic nitro dyes such as 4-(2′-hydroxyethyl)aminonitrotoluene, 2-amino-4,6-dinitrophenol, 2-amino-5-(2′-hydroxyethyl)aminonitrobenzene, 2-chloro-6-(ethylamino)-4-nitrophenol, 4-chloro-N-(2-hydroxyethyl)-2-nitroaniline, 5-chloro-2-hydroxy-4-nitroaniline, 2-amino-4-chloro-6-nitrophenol and 1-[(2′-ureidoethyl)amino-4-nitrobenzene, azo dyes such as 6-[(4′-aminophenyl)-azo]-5-hydroxynaphthalene-1-sulfonic acid sodium salt (C.I. 14 805), and dispersion dyes such as, for example, 1,4-diaminoanthraquinone and 1,4,5,8-tetraaminoanthraquinone. The colorant can contain these color components in an amount of from approximately 0.1 to 4 percent by weight.

Of course, additional coupler substances as well as developer substances and other color components can be used, where if they are bases, they can also be used in the form of their physiologically compatible salts with organic or inorganic acids such as, for example, hydrochloric acid or sulfuric acid, while if they possess aromatic OH-groups, they can also be used in the form of their salts with bases, for example, as alkali phenolates.

Moreover, if these colorants are to be used in the coloring of hair, they can contain further conventional cosmetic additives, for example, antioxidants such as ascorbic acid, thioglycolic acid, or sodium sulfite, as well as perfume oils, chelating agents, wetting agents, emulsifiers, thickeners, and conditioning agents.

The formulation of the colorant of the present invention can be, for example, a solution, especially an aqueous or aqueous alcoholic solution. However, especially preferred formulations are a creme, a gel, or an emulsion. The composition of the solution represents a mixture of dye components with the conventional additives for such formulations.

Conventional additives in solutions, cremes, emulsions, or gels are, for example, solvents such as water, lower aliphatic alcohols, for example, ethanol, propanol or isopropanol, glycerin or glycols such as 1,2-propylenglycol, and furthermore, wetting agents or emulsifiers from the anionic, cationic, amphoteric or nonionic classes of surface-active substances such as, for example, fatty alcohol sulfates, oxyethylated fatty alcohol sulfates, alkyl sulfonates, alkylbenzene sulfonates, alkyltrimethylammonium salts, alkyl betaines, oxyethylated fatty alcohols, oxyethylated nonylphenols, fatty acid alkanolamides and oxyethylated fatty acid esters, and moreover, thickeners such as higher fatty alcohols, starches, cellulose derivatives, petroleum jelly, paraffin oil and fatty acids, and conditioners such as cationic resins, lanolin derivatives, cholesterol, pantothenic acid, and betaine in addition. The above-mentioned components are used in the conventional amounts for such purposes, for example, wetting agents and emulsifiers in concentrations of from approximately 0.5 to 30 percent by weight, thickeners in an amount of from approximately 0.1 to 30 percent by weight, and conditioning agents in a concentration of from approximately 0.1 to 5 percent by weight.

Depending on the composition, the colorant of the present invention can be weakly acidic, neutral, or alkaline. In particular, it will exhibit a pH value of from 6.5 to 11.5, in which the pH can be adjusted to a basic value, preferably with ammonia. However, amino acids and/or organic amines, for example, monoethanolamine and triethanolamine, as well as inorganic bases such as, for example, sodium hydroxide and potassium hydroxide, can also be used. For adjusting the pH to a value in the acidic range, the use of inorganic or organic acids, for example, phosphoric acid, acetic acid, citric acid, or tartaric acid, can be considered.

For use in the oxidative coloring of hair, the above-described colorant is mixed with an oxidizing agent immediately before use, and an amount of this mixture that is sufficient for a hair coloring treatment, which is approximately 60 to 200 g depending on the relative thickness of the hair, is applied to the hair.

Hydrogen peroxide or its addition compounds with urea, melamine, sodium borate, or sodium carbonate in the form of a from 3 to 12%, preferably 6%, aqueous solution, and also oxygen in the air can all be given primary consideration as the oxidizing agent for developing the hair coloring. When a 6% hydrogen peroxide solution is used as the oxidizing agent, the weight ratio between the hair colorant and the oxidizing agent will be from 5:1 to 1:2, but preferably 1:1. Larger amounts of oxidizing agent are primarily used at higher dye concentrations or when a stronger bleaching of the hair is intended at the same time. The mixture is allowed to act on the hair at from 15 to 50° C. for from approx. 10 to 45 minutes, preferably for 30 minutes, after which the hair is rinsed with water and dried. Washing with shampoo can be combined with this rinsing as needed and, if necessary, a post-rinsing with a weak organic acid such as, for example, citric acid, or tartaric acid. Next the hair is dried.

A colorant of the present invention that contains o-aminophenol derivatives of Formula (1) enables coloring with excellent color fastness, especially with regard to lightfastness, color fastness during washing, and resistance to rubbing. With respect to color properties, the colorants of the present invention offer a broad palette of different color shades, depending on the type and composition of the color components, which extend from blonde through brown, crimson, and violet all the way to blue and black color tones. Thus, it is possible, for example, to achieve the hair colors of from blonde through brown through the use of a combination of compounds of the Formula (I) with 4-(2,5-diaminobenzylamino)aniline. In this case, the color tones obtained are characterized by their particular color intensity and a good color balance between damaged and undamaged hair. The very good color properties of the hair colorant of the present application are furthermore demonstrated by the fact that these agents enable the coloring of grayed hair, that has had no prior chemical damage, in a problem-free manner with good coverage.

The following examples will illustrate the object of the invention without limiting the invention to these examples.

### EXAMPLES

### Example 1

### Synthesis of o-aminophenol Derivatives of the General Formula (I) (General Synthesis Protocol without Protecting Groups, with a Large Excess of Amine)

A. Synthesis of 5-(2,2-dibromoethenyl)-2-nitrophenol

A solution of 7.9 g of triphenylphosphine in 70 ml of dry methylene chloride is added, drop by drop at 0° C., to a solution of 1.67 g of 3-hydroxy4-nitrobenzaldehyde and 5 g of carbon tetrabromide in 70 ml of dry methylene chloride. The reaction mixture is stirred at 0° C. for 1.5 hours, then the solvent is distilled off on a rotary evaporator, and the mixture is treated with 20 ml of chloroform. The precipitated product is filtered and the filtrate is evaporated. The crude product obtained is purified on silica gel with heptane/ethyl acetate (8: 1).

The yield was 2.0 g of 5-(2,2-dibromoethenyl)-2-nitrophenol obtained as a yellow powder.

¹H-NMR (300 MHz, DMSO): 10.52 (s, 1H, OH); 8.03 (d, J=9.0, 1H, H(3)); 7.39 (s, 1H, ethenyl H); 7.29 (d, J=1.8, 1H, H(6)); 7.05 (dd, J=1.8, J=9.0, 1H, H(4)).

B. Synthesis of 1-[(3-hydroxy-4-nitrophenyl)acetyllamine derivatives

Three ml of the corresponding amine is added to a solution of 0.48 g of the 5-(2,2-dibromoethenyl)-2-nitrophenol from step A in 1.5 ml of water. The reaction mixture is stirred at room temperature. At the completion of the reaction, the excess amine is distilled off on the rotary evaporator. The reaction mixture is treated with a 3N hydrochloric acid solution and extracted with methylene chloride. The combined organic phases are washed with a saturated NaCl solution and then dried with magnesium sulfate. The solvent is then distilled off on a rotary evaporator. It is possible to use the crude product obtained in the next step without further purification.

b1. 1-[(3-Hydroxy-4-nitrophenyl)acetyl]pyrrolidine

Amine used: pyrrolidine

Yield: 0.24 g

¹H-NMR (300 MHz. DMSO): 10.87 (s, 1H, OH); 7.84 (d, J=8.4, 1H, H(5)); 7.01 (d, J=1.8,1H, H(2)); 6.85 (dd, J=1.8, J=8.4, 1H, H(6)); 3.67 (s, 2H, CH2-C═O); 3.44 (t, 2H, J=6.6, N—CH2); 3.29 (t, 2H, J=6.6, N—CH2); 1.89-1.74 (m, 4H).

b2. 1-[(3-Hydroxy-4-nitrophenyl)acetyl]piperidine

Amine used: piperidine

Yield: 0.33 g

¹H-NMR (300 MHz. DMSO): 10.87 (s, 1H, OH); 7.84 (d, J=8.7, 1H, H(5)); 7.00 (d, J=1.8,1H, H(2)); 6.84 (dd, J=1.8, J=8.7, 1H, H(6)); 3.74 (s, 2H, CH2-C═O); 3.45-3.39 (m, 4H, N—CH2); 1.59-1.52 (m, 2H); 1.45-1.38 (m, 4H).

C. Synthesis of 1-[(4-amino-3-hydroxyphenyl)acetyllamine derivatives

A 0.8 mmol portion of 1-[(3-hydroxy4-nitrophenyl)acetyl]amine from step B is dissolved in 10 ml of tetrahydrofuran/ethanol 3:2, and this is stirred during the addition of 20 mg of a palladium-activated carbon-catalyst (10%) and 120 mg of hydrazine hydrate at 25° C.

At the completion of the reaction, the catalyst is filtered off through Celite and washed with ethanol. After concentration of the solution on a rotary evaporator, the crude product is dried.

c1. 1-[(4-Amino-3-hydroxyphenyl)acetyl]pyrrolidine

Amine used: 1-[(3-hydroxy4-nitrophenyl)acetyl]pyrrolidine (b1)

Yield: 0.13 g

¹H-NMR (300 MHz, DMSO): 8.92 (s, 1H, OH); 6.56 (d, J=1.5, 1H, H(2)); 6.49 (d, J=7.8,1H, H(5)); 6.40 (dd, J=1.5, J=7.8, 1H, H(6)); 4.35 (s, 2H, NH2); 3.33 (s, 2H, CH2-C═O); 3.41-3.23 (m, 4H, N—CH2); 1.84-1.71 (m, 4H).

ESI-MS: 243 [M+Na]⁺ (100)

c2. 1-[(4-Amino-3-hydroxyphenyl)acetyl]piperidine

Amine used: 1-[(3-Hydroxy-4-nitrophenyl)acetyl]piperidine (b2)

Yield: 0.14 g

¹H-NMR (300 MHz DMSO): 9.00 (s, 1H, OH); 6.55 (d, J=1.8, 1H, H(2)); 6.50 (d, J=7.8,1H, H(5)); 6.41 (dd, J=1.8, J=7.8, 1H, H(6)); 4.36 (s, 2H, NH2); 3.42 (s, 2H, CH2-C═O); 3.42-3.33 (m, 4H, N—CH2); 1.51-1.49 (m, 2H); 1.48-1.37 (m, 2H); 1.34-1.27 (m, 2H).

ESI-MS: 257 [M+Na]⁺ (100)

### Example 2

### Synthesis of o-aminophenol Derivatives of the General Formula (I) (General Synthesis Protocol with Protecting Groups)

D. Synthesis of 4-nitro-3-[( phenylmethyl)oxy]benzaldehyde

2.88 g of a sodium hydride dispersion (55% in oil) is added, portion by portion at 0° C., to a solution of 10.32 g of 3-hydroxy-4-nitrobenzaldehyde in 180 ml dry dimethylacetamide. The reaction mixture is then stirred for 30 minutes at 0° C. 10.8 g of benzyl bromide is added, drop by drop, to this mixture, and the reaction mixture is then stirred for 30 minutes, followed by 30 minutes at room temperature, and then for 2 hours at 70° C. Subsequently, the reaction mixture is treated with 900 ml of an ice/water mixture and then stirred for another 1 hour. The mixture is filtered, and the precipitate is washed with water and then dried.

The yield obtained is 15.68 g of 4-nitro-3-[(phenylmethyl)oxy]benzaldehyde.

The crude product obtained is used in the next step without further purification.

¹H-NMR (300 MHz, DMSO): 10.07 (s, 1H, COH); 8.10 (d, J=8.1, 1H, H(5)); 7.93 (d, J=1.5,1H, H(2)); 7.68 (dd, J=1.5, J=8.1, 1H, H(6)); 7.48-7.32 (m, 5H, phenyl); 5.41 (s, 2H, CH2-Phenyl).

E. Synthesis of 4-(2,2-dibromethenyl)-1-nitro-2-1(phenylmethyl)oxy]benzene

A solution of 44.5 g triphenylphosphine in 150 ml dry methylene chloride is added, drop by drop 0° C., to a solution of 15.3 g of 4-nitro-3-[(phenylmethyl)oxy]benzaldehyde from step D and 28.1 g of carbon tetrabromide in 300 ml of dry methylene chloride. The reaction mixture is stirred for 1.5 hours at 0° C. Next, the solvent is distilled off on a rotary evaporator and the residue is treated with 120 ml of chloroform. The precipitated product is filtered and the filtrate is evaporated. The crude product obtained is purified on silica gel with heptane/ethyl acetate (3:1).

This procedure yielded 19 g of 4-(2,2-dibromethenyl)-1-nitro-2-[(phenylmethyl)oxy]benzene as a yellow powder.

¹H-NMR (300 MHz, DMSO): 7.95 (d, J=8.4, 1H, H(6)); 7.87 (s, 1H, H ethenyl); 7.63 (d, J=1.2,1H, H(3)); 7.48-7.36 (m, 5H, phenyl); 7.34 (dd, J=1.2, J=8.4, 1H, H(5)); 5.32 (s, 2H, CH2-phenyl).

F. Synthesis of 1-{4-nitro-3-[(phenylmethyl)oxy]phenyl}acetyl)amine derivatives

0.22 g of potassium hydroxide and 1.7 mmol of the corresponding amine are added to a solution of 0.41 g of 4-(2,2-dibromethenyl)-1-nitro-2-[(phenylmethyl)oxy]benzene from step E in 4 ml of tetrahydrofuran and 3 ml of water. The reaction mixture is stirred at room temperature. After completion of the reaction, the reaction mixture is treated with a 1N hydrochloric acid solution and extracted with ethyl acetate. The combined organic phases are washed with a saturated NaCl solution and then dried with magnesium sulfate. The solvent is distilled off on a rotary evaporator and the residue is purified on silica gel.

f1. 1-({4-Nitro-3-[(phenylmethyl)oxy]phenyl}acetyl)pyrrolidine

Amine used: pyrrolidine

Yield: 0.23 g

1H-NMR (300 MHz DMSO): 7.84 (d, J=8.4, 1H, H(5)); 7.49-7.34 (m, 5H, phenyl); 7.33 (d, J=1.5,1H, H(2)); 6.98 (dd, J=1.5, J=8.4, 1H, H(6)); 5.28 (s, 2H, CH2-phenyl); 3.73 (s, 2H, CH2-C═O); 3.44 (t, 2H, J=6.6, N—CH2); 3.29 (t, 2H, J=6.6, N—CH42); 1.89-1.74 (m, 4H).

f2. ]-({4-Nitro-3-[(phenylmethyl)oxy]phenyl}acetyl)morpholine

Amine used: morpholine

Yield: 0.22 g

1H-NMR (300 MHz, DMSO): 7.85 (d, J=8.4, 1H, H(5)); 7.49-7.35 (m, 5H, phenyl); 7.33 (d, J=1.5,1H, H(2)); 6.98 (dd, J=1.5, J=8.4, 1H, H(6)); 5.28 (s, 2H, CH2-phenyl); 3.82 (s, 2H, CH2-C═O); 3.54-3.43 (m, 8H).

f3. 1-({4-Nitro-3-[(phenylmethyl)oxy]phenyl}acetyl)piperidine

Amine used: piperidine

Yield: 0.21 g

¹H-NMR (300 MHz, DMSO): 7.85 (d, J=8.4, 1H, H(5)); 7.49-7.35 (m, 5H, phenyl); 7.33 (d, J=1.5,1H, H(2)); 6.98 (dd, J=1.5, J=8.4, 1H, H(6)); 5.28 (s, 2H, CH2-phenyl); 3.80 (s, 2H, CH2-C═O); 3.45-3.39 (m, 4H, N—CH2); 1.59-1.52 (m, 2H); 1.45-1.38 (m, 4H).

f4. 1-({4-Nitro-3-[(phenylmethyl)oxy]phenyl}acetyl)-4-piperidinol

Amine used: 4-piperidinol

Yield: 0.20 g

1H-NMR (300 MHz. DMSO): 7.85 (d, J=8.4, 1H, H(5)); 7.49-7.35 (m, 5H, phenyl); 7.33 (d, J=1.5,1H, H(2)); 6.98 (dd, J=1.5, J=8.4, 1H, H(6)); 5.28 (s, 2H, CH2-phenyl); 4.74 (d, J=4.2, 1H, OH); 3.92-3.86 (m, 1H, CH—OH); 3.81 (s, 2H, CH2-C═O); 3.73-3.63 (m, 2H); 3.19-3.11 (m, 1H); 3.05-2.97 (m, 1H); 1.68-1.64 (m, 2H); 1.29-1.17 (m, 2H).

f5. N,N-Diethyl-2-{4-nitro-3-[(phenylmethyl)oxy]phenyl}acetamide

Amine used: diethylamine

Yield: 0.19 g

1H-NMR (300 MHz, DMSO): 7.85 (d, J=8.4, 1H, H(5)); 7.49-7.34 (m, 5H, phenyl); 7.33 (d, J=1.5,1H, H(2)); 6.98 (dd, J=1.5, J=8.4, 1H, H(6)); 5.28 (s, 2H, CH2-phenyl); 3.37-3.24 (m, 4H, N—CH2); 3,31 (s, 2H, CH2-C═O); 1.08 (t, J=6.9, 3H, CH3); 1.01 (t, J=6.9, 3H, CH3).

f6. 2-{4-Nitro-3-[(phenylmethyl)oxy]phenyl}-N-propylacetamide

Amine used: propylamine

Yield: 0.09 g

¹H-NMR (300 MHz, DMSO): 8.1 (m, 1H, NH); 7.85 (d, J=8.4, 1H, H(5)); 7.49-7.35 (m, 5H, phenyl); 7.37 (d, J=1.5,1H, H(2)); 7.00 (dd, J=1.5, J=8.4, 1H, H(6)); 5.28 (s, 2H, CH2-phenyl); 3.50 (s, 2H, CH2-C═O); 3.01 (q, J=7.2, 2H, NH-CH2); 1.41 (q, J=7.2, 2H, CH2-CH3); 0.84 (t, J=7.2, 3H, CH3).

ESI-MS: 351 [M+Na]⁺ (100)

f7. 2-{4-Nitro-3-[(phenylmethyl)oxy]phenyl}-N-(tetrahydro-2-furanylmethyl)acetamide

Amine used: tetrahydrofiurfurylamine

Yield: 0.116 g

1H-NMR (300 MHz, DMSO): 8.22 (t, J=5.37, 1H, NH); 7.85 (d, J=8.4, 1H, H(5)); 7.49-7.33 (m, 5H, phenyl); 7.37 (d, J=1.5,1H, H(2)); 7.01 (dd, J=1.5, J=8.4, 11H, H(6)); 5.27 (s, 2H, CH2-phenyl); 3.87-3.71 (m, 2H); 3.64-3.57 (m, 1H); 3.54 (s, 2H, CH2-C═O); 3.21-3.05 (m, 2H); 1.88-1.75 (m, 2H); 1.51-1.42 (m, 1H); 1.23-1.14 (m, 1H).

ESI-MS: 393 [M+Na]⁺ (100)

G. Synthesis of 1-[(4-amino-3-hydroxyphenyl)acetyl]amine derivatives

A 0.8 mmol portion of 1-{4-nitro-3-[(phenylmethyl)oxy]phenyl}acetyl)amine from step F is dissolved in 10 ml tetrahydrofuiran/ethanol 3:2, and after addition of 27 mg of a palladium-activated, carbon-catalyst (10%), this mixture is hydrogenated. After taking up the required amount of hydrogen, the reaction mixture is freed from the catalyst by filtration, and treated with an equivalent of concentrated phosphoric acid as needed. After concentration of the solution on a rotary evaporator, the residue is dried at 40° C.

The product is re-crystallized from methanol or a methanol/ethyl acetate mixture as needed.

g1. 1-[(4-Amino-3-hydroxyphenyl)acetyl]pyrrolidine phosphate

Amine used: 1-({4-nitro-3-[(phenylmethyl)oxy]phenyl}acetyl)pyrrolidine (f1)

Yield: 0.15 g

1H-NMR (300 MHz DMSO): 7.9-7.0 (s, broad, 4H, OH and NH3+); 6.56 (d, J=1.8,1H, H(2)); 6.50 (d, J=8.1, 1H, H(5)); 6.41 (dd, J=1.8, J=8.1, 1H, H(6)); 3.35 (s, 2H, C1H2-C═O); 3.41 (t, 2H, J=6.6, N—CH2); 3.26 (t, 2H, J-6.6, N—CH2); 1.89-1.69 (m, 4H).

ESI-MS: 243 [M+Na]⁺ (100)

g2. 1-[(4-Amino-3-hydroxyphenyl)acetyl]morpholine

Amine used: 1-({4-nitro-3-[(phenylmethyl)oxy]phenyl}acetyl)morpholine (f2)

Yield: 0.18 g

1H-NMR (300 MHz, DMSO0: 8.96 (s, broad, 1H, OH); 6.54 (d, J=1.8,1H, H(2)); 6.50 (d, J=7.8, 1H, H(5)); 6.40 (dd, J=1.8, J=7.8, 1H, H(6)); 4.37 (s, broad, 2H, NH2); 3.33 (s, 2H, CH2-C═O); 3.51-3.41 (m, 8H).

ESI-MS: 259 [M+Na]⁺ (100)

g3. -[(4-Amino-3-hydroxyphenyl)acetyl]piperidine

Amine used: 1-({4-nitro-3-[(phenylmethyl)oxy]phenyl}acetyl)piperidine (f3)

Yield: 0.18 g

¹H-NMR (300 MHz DMSO): 9.00 (s, broad, 1H, OH); 6.55 (d, J=1.8, 1H, H(2)); 6.50 (d, J=7.8,1H, H(5)); 6.41 (dd, J=1.5, J=7.8, 1H, H(6)); 4.36 (s, 2H, NH2); 3.42 (s, 2H, CH2-C═O); 3.42-3.33 (m, 4H, N—CH2); 1.51-1.49 (m, 2H); 1.48-1.37 (m, 2H); 1.34-1.27 (m, 2H).

ESI-MS: 257 [M+Na]⁺ (100)

g4. 1-[(4-Amino-3-hydroxyphenyl)acetyl]-4-piperidinol

Amine used: 1-({4-nitro-3-[(phenylmethyl)oxy]phenyl}acetyl)-4-piperidinol (f4)

Yield: 0.19 g

1H-NMR (300 MHz DMSO): 8.94 (s, broad, 1H, OH); 6.54 (d, J=1.2,1H, H(2)); 6.50 (d, J=7.8, 1H, H(5)); 6.41 (dd, J=1.2, J=7.8, 1H, H(6)); 4.68 (d, J=3.3, 1H, OH); 4.35 (s, broad, NH2); 3.93-3.88 (m, 1H, CH—OH); 3.68-3.60 (m, 2H); 3.33 (s, 2H, CH2-C═O); 3.12-3.03 (m, 1H); 2.99-2.90 (m, 1H); 1.65-1.54 (m, 2H); 1.29-1.03 (m, 2H).

ESI-MS: 273 [M+Na]⁺ (100)

g5. 2-(4-Amino-3-hydroxyphenyl)-N,N-diethylacetamide phosphate

Amine used: N,N-diethyl-2-{4-nitro-3-[(phenylmethyl)oxy]phenyl}acetamide (f5)

Yield: 0.19 g

¹H-NMR (300 MHz. DMSO): 7.9-6.7 (s, broad, 4H, NH3+and OH); 6.57 (s, 1H, H(2)); 6.51 (d, J=7.8, 1H, H(5)); 6.41 (d, J=7.8, 1H, H(6)); 3.40 (s, 2H, CH2-C═O); 3.30-3.20 (m, 4H, N—CH2); 1.05-0.97 (m, 6H, CH3).

ESI-MS: 253 [M+Na]⁺ (100)

g6. 2-(4-Amino-3-hydroxyphenyl)-N-propylacetamide phosphate

Amine used: 2-{4-nitro-3-[(phenylmethyl)oxy]phenyl}-N-propylacetamide (f6)

Yield: 0.20 g

1H-NMR (300 MHz, DMSO): 9.4-8.4 (s, broad, 1H, OH); 7.78 (t, J=7.2, NH-CH2); 6.58 (d, J=1.5, 1H, H(2)); 6.50 (d, J=7.8, 1H, H(5)); 6.42 (dd, J=1.5, J=7.8, 1H, H(6)); 6.3-5.3 (s, broad, 4H, OH and NH13+); 3.13 (s, 2H, CH2-C═O); 2.97 (q, J=7.2, 2H, NH-CH2); ); 1.38 (sext, J=7.2, 2H, CH2-CH3); 0.82 (t, J=7.2, 3H, CH3).

ESI-MS: 231 [M+Na]⁺ (100)

g7. 2-(4-Amino-3-hydroxyphenyl)-N-(tetrahydro-2-furanylmethyl)acetamide

Amine used: 2-{4-nitro-3-[(phenylmethyl)oxy]phenyl}-N-(tetrahydro-2-furanylmethyl)acetamide (f7)

Yield: 0.15 g

1H-NMR (300 MHz DMSO): 8.9 (s, broad, 1H, OH); 7.80 (t, NH-CH2); 6.57 (d, J=1.8, 1H, H(2)); 6.48 (d, J=7.8, 1H, H(5)); 6.42 (dd, J=1.8, J=7.8, 1H, H(6)); 4.35 (s, broad, 2H, NH2); 3.83-3.69 (m, 2H); 3.60-3.42 (m, 1H); 3.13 (s, 2H, CH2-C═O); 3.11-3.01 (m, 2H); 1.9-1.75 (m, 1H); 1.50-1.37 (m, 1H).

ESI-MS: 273 [M+Na]⁺ (100)

### Examples 3 through 39

### Hair Colorant

Hair coloring solutions of the following composition are prepared:

| | | |
|---|---|---|
| X | g | o-aminophenol of Formula (I), G1 through |
| | | G7, according to Table 1 |
| U | g | developer substances E8 through E15 |
| | | according to Table 2 |
| Y | g | coupler substances K12 through K35 |
| | | according to Table 4 |
| Z | g | direct-penetrating dyes D1 through D3 |
| | | according to Table 3 |
| 10.000 | g | lauryl ether sulfate (28% aqueous solution) |
| 9.000 | g | ammonia (22% aqueous solution) |
| 7.800 | g | ethanol |
| 0.300 | g | ascorbic acid |
| 0.300 | g | ethylenediaminetetraacetic acid disodium |
| | | salt hydrate |
| balanced to 100.000 | g | with water |

A 10 g portion of the above dye solution is mixed with 10 g of a 6% aqueous hydrogen peroxide solution immediately before use. Then the mixture is applied to bleached hair. After an action period of 30 minutes at 40° C., the hair is rinsed with water, washed with a commercially available shampoo, and dried. The coloring results are compiled in Table 6.

### Examples 40 through 51

### Hair Colorant

Creme-type color vehicles of the following compositions were prepared:

| | | |
|---|---|---|
| X | g | o-aminophenol of Formula (I), G1/G6 |
| | | according to Table 1 |
| U | g | developer substance E8 through E15 |
| | | according to Table 2 |
| Y | g | coupler substance K12 through K31 |
| | | according to Table 4 |
| Z | g | direct-penetrating dye D2 according to Table 3 |
| 15.0 | g | cetyl alcohol |
| 0.3 | g | ascorbic acid |
| 3.5 | g | sodium lauryl alcohol diglycol ether sulfate, |
| | | 28% aqueous solution |
| 3.0 | g | ammonia 22% aqueous solution |
| 0.3 | g | sodium sulfite, anhydrous |
| balanced to 100.0 | g | with water |

A 10 g portion of the above color creme was mixed with 10 g of a 6% hydrogen peroxide solution immediately before use. Then, the mixture is applied to the hair. After an action period of 30 minutes at room temperature, the hair is rinsed with water, washed with a commercially available shampoo, and dried. The coloring results are compiled in Table 7.

### Examples 52 to 59

### Hair Colorant

Color vehicles of the following composition are prepared:

| | | |
|---|---|---|
| X | g | o-aminophenol of Formula (I), G6 |
| | | according to Table 1 |
| Z | g | oxidative dye W1/W2 according to Table 5 |
| U | g | developer substance E8 through E15 |
| | | according to Table 2 |
| 10.000 | g | lauryl ether sulfate (28% aqueous solution) |
| 9.000 | g | ammonia (22% aqueous solution) |
| 7.800 | g | ethanol |
| 0.300 | g | ascorbic acid |
| 0.300 | g | ethylenediaminetetraacetic acid disodium |
| | | salt hydrate |
| balanced to 100.000 | g | with water |

A 10 g portion of the above dye solution is mixed with 10 g of a 6% hydrogen peroxide solution immediately before use. Then, the mixture is applied to bleached hair. After an action period of 30 minutes at 40° C., the hair is rinsed with water, washed with a commercially available shampoo, and then dried. The coloring results are compiled in Table 8.

Unless otherwise indicated, all percent values in the present application refer to percent by weight.

TABLE 1
o-Aminophenol derivatives of Formula (I)

| | |
|---|---|
| G1 | 1-[(4-amino-3-hydroxyphenyl)acetyl]pyrrolidine phosphate |
| G2 | 1-[(4-amino-3-hydroxyphenyl)acetyl]morpholine |
| G3 | 1-[(4-amino-3-hydroxyphenyl)acetyl]piperidine |
| G4 | 1-[(4-amino-3-hydroxyphenyl)acetyl]-4-piperidinol |
| G5 | 2-(4-amino-3-hydroxyphenyl)-N,N-diethylacetamide phosphate |
| G6 | 2-(4-amino-3-hydroxyphenyl)-N-propylacetamide phosphate |
| G7 | 2-(4-amino-3-hydroxyphenyl)-N-(tetrahydro-2- |
| | furanylmethyl)acetamide |

TABLE 2
Developer substances

| | |
|---|---|
| E8 | 1,4-diaminobenzene |
| E9 | 2,5-diaminophenylethanol sulfate |
| E10 | 3-methyl-4-aminophenol |
| E11 | 4-amino-2-aminomethylphenol dihydrochloride |
| E12 | 4-aminophenol |
| E14 | 4,5-diamino-1-(2′-hydroxyethyl)pyrazole sulfate |
| E15 | 2,5-diaminotoluene sulfate |

TABLE 3
Direct-penetrating dyes

| | |
|---|---|
| D1 | 2,6-diamino-3-((pyridin-3-yl)azo)pyridine |
| D2 | 6-chloro-2-ethylamino-4-nitrophenol |
| D3 | 2-amino-6-chloro-4-nitrophenol |

TABLE 4
Coupler substances

| | |
|---|---|
| K12 | 2-amino-4-(2′-hydroxyethyl)aminoanisole sulfate |
| K13 | 1,3-diamino-4-(2′-hydroxyethoxy)benzene sulfate |
| K16 | 3,5-diamino-2,6-dimethoxypyridine dihydrochloride |
| K17 | 2,4-diamino-5-ethoxytoluene sulfate |
| K18 | N-(3-dimethylamino)phenylurea |
| K21 | 3-aminophenol |
| K22 | 5-amino-2-methylphenol |
| K23 | 3-amino-2-chloro-6-methylphenol |
| K25 | 1-naphthol |
| K26 | 1-acetoxy-2-methylnaphthalene |
| K31 | 1,3-dihydroxybenzene |
| K32 | 2-methyl-1,3-dihydroxybenzene |
| K33 | 1-chloro-2,4-dihydroxybenzene |
| K35 | 3,4-methylenedioxyphenol |

TABLE 5
Oxidative dyes

| | |
|---|---|
| W1 | 4-(2,5-diaminobenzylamino)aniline hydrochloride |
| W2 | 2-(3-amino-phenyl)aminomethyl-1,4-diaminobenzene |
| | hydrochloride |

TABLE 6
Hair colorant
Example No.

| | 3 | 4 | 5 | 6 | 7 | 8 | 9 |
|---|---|---|---|---|---|---|---|
| Dyes | (Dye amount in grams) | | | | | | |
| G1 | 0.30 | | | | | | |
| G2 | | 0.30 | | | | | |
| G3 | | | 0.30 | | | | |
| G4 | | | | 0.30 | | | |
| G5 | | | | | 0.30 | | |
| G6 | | | | | | 0.30 | |
| G7 | | | | | | | 0.30 |
| Coloring | bright | bright | bright | bright | bright | bright | bright |
| results | yellow | yellow | yellow | yellow | yellow | yellow | yellow |

Example No.

| | | | | | |
|---|---|---|---|---|---|
| 10 | 11 | 12 | 13 | 14 | 15 |

| | |
|---|---|
| Dyes | (Dye amount in grams) |

| | | | | | | |
|---|---|---|---|---|---|---|
| G1 | 0.03 | | | | 0.05 | |
| G3 | | 0.03 | | | | |
| G5 | | | 0.03 | | | |
| G6 | | | | 0.03 | | 0.05 |
| E11 | 0.55 | 0.55 | 0.55 | 0.55 | | |
| E12 | | | | | 0.55 | 0.55 |
| K25 | 0.30 | 0.30 | 0.30 | 0.30 | | |
| K26 | | | | | 0.35 | 0.35 |
| K32 | 0.22 | 0.22 | 0.22 | 0.22 | | |
| K33 | | | | | 0.20 | 0.20 |
| Coloring | auburn | auburn | auburn | auburn | auburn | Auburn |
| results | | | | | | |

Example No.

| | | | |
|---|---|---|---|
| 16 | 17 | 18 | 19 |

| | |
|---|---|
| Dyes | (Dye amount in grams) |

| | | | | |
|---|---|---|---|---|
| G1 | 0.03 | 0.02 | | |
| G6 | | | 0.03 | 0.02 |
| E10 | 0.55 | | 0.55 | |
| E14 | | 0.55 | | 0.55 |
| K25 | 0.30 | 0.30 | 0.30 | 0.30 |
| K26 | | | | |
| K31 | 0.18 | 0.20 | 0.18 | 0.20 |
| Coloring | auburn | auburn | auburn | auburn |
| results | | | | |

Example No.

| | | | |
|---|---|---|---|
| 20 | 21 | 22 | 23 |

| | |
|---|---|
| Dyes | (Dye amount in grams) |

| | | | | |
|---|---|---|---|---|
| G1 | 0.01 | 0.005 | | |
| G6 | | | 0.01 | 0.005 |
| E8 | 0.10 | 0.10 | 0.10 | 0.10 |
| E9 | 0.25 | | 0.25 | |
| E15 | | 0.25 | | 0.25 |
| K13 | 0.09 | 0.09 | 0.09 | 0.09 |
| K21 | 0.05 | | 0.05 | |
| K22 | | 0.05 | | 0.05 |
| K31 | 0.20 | | 0.20 | |
| K32 | | 0.20 | | 0.20 |
| Coloring | blonde | blonde | blonde | blonde |
| results | | | | |

| | Example No. | | | | | |
|---|---|---|---|---|---|---|
| | 24 | 25 | 26 | 27 | 28 | 29 |
| Dyes | (Dye amount in grams) | | | | | |
| G1 | 0.01 | 0.006 | 0.02 | 0.005 | 0.05 | 0.01 |
| E9 | | | | | 0.096 | 1.80 |
| E10 | 0.096 | 0.24 | 0.30 | 0.90 | 0.01 | 0.70 |
| K12 | | | | | 0.01 | |
| K18 | | | | | | 0.03 |
| K21 | | | | | 0.02 | 0.06 |
| K22 | 0.08 | 0.2 | 0.25 | 0.056 | | 0.58 |
| K25 | | | | | 0.03 | |
| K31 | | | | 0.20 | | 0.80 |
| K32 | | 0.03 | 0.05 | 0.316 | | |
| K35 | 0.018 | | | | | |
| D2 | | | | 0.01 | | |
| D3 | 0.04 | 0.06 | 0.025 | | | |
| Coloring | light blonde | copper gold | light copper | crimson | silver blonde | dark |
| results | to copper | | colors | brown | | mahogany |
| | gold | | | | | |
| | Example No. | | | | | |
| | 30 | 31 | 32 | 33 | 34 | 35 |
| Dyes | (Dye amount in grams) | | | | | |
| G6 | 0.01 | 0.006 | 0.02 | 0.005 | 0.05 | 0.01 |
| E9 | | | | | 0.096 | 1.80 |
| E10 | 0.096 | 0.24 | 0.30 | 0.90 | 0.01 | 0.70 |
| K12 | | | | | 0.01 | |
| K18 | | | | | | 0.03 |
| K21 | | | | | 0.02 | 0.06 |
| K22 | 0.08 | 0.20 | 0.25 | 0.056 | | 0.58 |
| K25 | | | | | 0.03 | |
| K31 | | | | 0.20 | | 0.80 |
| K32 | | 0.03 | 0.05 | 0.316 | | |
| K35 | 0.018 | | | | | |
| D2 | | | | 0.01 | | |
| D3 | 0.04 | 0.06 | 0.025 | | | |
| Coloring | light blonde | copper gold | light copper | crimson | silver blonde | dark |
| results | to copper | | colors | brown | | mahogany |
| | gold | | | | | |

Example No.

| | | | |
|---|---|---|---|
| 36 | 37 | 38 | 39 |

| | |
|---|---|
| Dyes | (Dye amount in grams) |

| | | | | |
|---|---|---|---|---|
| G1 | 0.03 | | 0.15 | |
| G6 | | 0.03 | | 0.15 |
| E14 | 0.05 | 0.05 | 0.10 | 0.10 |
| E8 | | | 0.50 | 0.50 |
| E10 | 0.60 | 0.60 | 0.05 | 0.5 |
| K12 | | | 1.10 | 1.10 |
| K17 | | | 1.10 | 1.10 |
| K22 | 0.50 | 0.50 | | |
| K23 | | | 0.60 | 0.60 |
| K32 | 0.03 | 0.03 | | |
| D1 | 0.25 | 0.25 | | |
| D2 | | | 0.50 | 0.50 |
| D3 | 0.15 | 0.15 | | |
| Coloring | orange | orange | red-orange | Red-orange |
| results | | | | |

TABLE 7
hair colorant

| | Example No. | | | | | |
|---|---|---|---|---|---|---|
| | 40 | 41 | 42 | 43 | 44 | 45 |
| Dyes | (Dye amount in grams) | | | | | |
| G1 | 0.10 | 0.05 | 0.01 | | | |
| G6 | | | | 0.10 | 0.05 | 0.01 |
| E15 | 0.70 | 0.70 | 0.70 | 0.70 | 0.70 | 0.70 |
| K12 | 0.10 | 0.10 | 0.10 | 0.10 | 0.10 | 0.10 |
| K13 | | | | | | |
| K23 | 0.10 | 0.10 | 0.10 | 0.10 | 0.10 | 0.10 |
| K31 | 0.40 | 0.40 | 0.40 | 0.40 | 0.40 | 0.40 |
| D2 | 0.10 | 0.10 | 0.10 | 0.10 | 0.10 | 0.10 |
| Coloring | brown | brown | brown | brown | brown | brown |
| results | | | | | | |

| | Example No. | | | | | |
|---|---|---|---|---|---|---|
| | 46 | 47 | 48 | 49 | 50 | 51 |
| Dyes | (Dye amount in grams) | | | | | |
| G1 | 0.005 | 0.27 | 0.01 | | | |
| G6 | | | | 0.005 | 0.27 | 0.01 |
| E8 | 0.25 | | | 0.25 | | |
| E9 | | 1.71 | 0.02 | | 1.71 | 0.02 |
| E10 | 2.00 | 0.20 | 0.01 | 2.00 | 0.20 | 0.01 |
| K13 | | 0.10 | | | 0.10 | |
| K16 | | | 0.015 | | | 0.015 |
| K21 | | 0.80 | | | 0.80 | |
| K22 | 1.80 | | 0.25 | 1.80 | | 0.25 |
| K23 | | 0.20 | | | 0.20 | |
| K26 | | | 0.03 | | | 0.03 |
| K31 | 0.25 | 0.135 | 0.02 | 0.25 | 0.135 | 0.02 |
| D2 | | 0.01 | | | 0.01 | |
| Color | orange | chocolate | silver | orange | chocolate | silver |
| tone | colors | brown | blonde | colors | brown | blonde |

TABLE 8

Example No.

| | | | |
|---|---|---|---|
| 52 | 53 | 54 | 55 |

| | |
|---|---|
| Dyes | (Dye amount in grams) |

| | | | | |
|---|---|---|---|---|
| G6 | 0.01 | 0.18 | 0.04 | 0.18 |
| E8 | | 0.12 | | 0.12 |
| E9 | | | 0.12 | |
| E15 | | | | |
| W1 | 0.90 | | | 0.38 |
| W2 | | 0.37 | 0.05 | |
| Coloring | deep blue | medium | medium | black-brown |
| results | | brown | blonde | |

Example No.

| | | | |
|---|---|---|---|
| 56 | 57 | 58 | 59 |

| | |
|---|---|
| Dyes | (Dye amount in grams) |

| | | | | |
|---|---|---|---|---|
| G6 | 0.18 | 0.18 | 0.06 | 0.18 |
| E8 | | | | |
| E9 | 0.15 | | | |
| E15 | | 0.13 | | |
| W1 | | 0.38 | 0.38 | 0.38 |
| W2 | 0.58 | | | |
| Coloring | brown | black- | medium | brown |
| results | | brown | brown | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An o-Aminophenol derivative of Formula (I), or physiologically compatible water-soluble salts thereof, 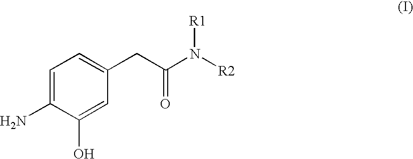
wherein
R1 and R2, are substituents independently selected from the group consisting of: hydrogen, a saturated or unsaturated (C₁-C₆) alkyl group, a (C₁-C₆) hydroxyalkyl group, a (C₂-C₆) dihydroxyalkyl group, a (C₁-C₃)-alkoxy-( Cl-C₃)-alkyl group, a (C₁-C₃)-hydroxyalkyl-(C₁-C₃)-alkoxy group, a (C₁-C₆) aminoalkyl group, a (C₁-C₄)-alkylamine-(C₁-C₄)-alkyl group, a di-(C₁-C₄)-alkylamino-(C₁-C₄)-alkyl group, a (C₁-C₆) acetylaminoalkyl group, a (C₁-C₆) cyanoalkyl group, a (C₁-C₆) carboxyalkyl group, a (C₁-C₆) aminocarbonylalkyl group, an unsubstituted or substituted phenyl group, a benzyl group, a furfuryl group, a tetrahydrofurfuryl group or a pyridylmethyl group, or R1 and R2 together with the N-atom can optionally form a substituted, saturated or unsaturated about 4-membered to about 8-membered ring, which optionally comprises an additional heteroatom.

2. An o-Aminophenol derivative according to claim 1, wherein R1 and R2 are selected from the group consisting of hydrogen, a substituted or unsubstituted (C₁-C₆) alkyl group.

3. An o-Aminophenol derivative according to claim 1, wherein R1 and R2 form a substituted, saturated about 4-membered to about 8-membered ring, which optionally comprises an additional O-, S-, or N-atom.

4. An o-aminophenol derivative according to claim 1, wherein it is selected from the group consisting of 1-[(4-amino-3-hydroxyphenyl)acetyl]pyrrolidine, 1-[(4-amino-3-hydroxyphenyl)acetyl]piperidine, 1-[(4-amino-3-hydroxyphenyl)acetyl]4-hydroxypiperidine, 1-[(4-amino-3-hydroxyphenyl)acetyl]morpholine, 1-[(4-amino-3-hydroxyphenyl)acetyl]piperazine, 1-[(4-amino-3-hydroxyphenyl)acetyl]-4-methylpiperazine, 2-(4-amino-3-hydroxyphenyl)acetamide, 2-(4-amino-3-hydroxyphenyl)-N-methylacetamide, 2-(4-amino-3-hydroxyphenyl)-N-ethylacetamide, 2-(4-amino-3-hydroxyphenyl)-N-propylacetamide, 2-(4-amino-3-hydroxyphenyl)-N-butylacetamide, 2-(4-amino-3-hydroxyphenyl)-N-(2-hydroxyethyl)acetamide, 2-(4-amino-3-hydroxyphenyl)-N-(3-hydroxypropyl)acetamide, 2-(4-amino-3-hydroxyphenyl)-N-(4-hydroxybutyl)acetamide, 2-(4-amino-3-hydroxyphenyl)-N-(2,3-dihydroxypropyl)acetamide, 2-(4-amino-3-hydroxyphenyl)-N-[2-(methyloxy)ethyl]acetamide, 2-(4-amino-3-hydroxyphenyl)-N-[3-(methyloxy)propyl]acetamide, 2-(4-amino-3-hydroxyphenyl)-N-{2-[(2-hydroxyethyl)oxy]ethyl}acetamide, 2-(4-amino-3-hydroxyphenyl)-N-(cyanomethyl)acetamide, 2-(4-amino-3-hydroxyphenyl)-N-(2-aminoethyl)acetamide, 2-(4-amino-3-hydroxyphenyl)-N-(3-aminopropyl)acetamide, 2-(4-amino-3-hydroxyphenyl)-N,N-dimethylacetamide, 2-(4-amino-3-hydroxyphenyl)-N,N-diethylacetamide, 2-(4-amino-3-hydroxyphenyl)-N,N-dipropylacetamide, 2-(4-amino-3-hydroxyphenyl)-N,N-diisopropylacetamide, 2-(4-amino-3-hydroxyphenyl)-N,N-dibutylacetamide, 2-(4-amino-3-hydroxyphenyl)-N,N-bis-(2-hydroxyethyl)acetamide, 2-(4-am ino-3-hydroxyphenyl)-N,N-bis-(3-hydroxypropyl)acetamide, 2-(4-amino-3-hydroxyphenyl)-N-(tetrahydro-2-furanylmethyl)acetamide and 2-(4-amino-3-hydroxyphenyl)-N-(2-furanylmethyl)acetamide, 2-(4-amino-3-hydroxyphenyl)-N-phenylacetamide, 2-(4-amino-3-hydroxyphenyl)-N-(4-hydroxyphenyl)acetamide, 2-(4-amino-3-hydroxyphenyl)-N-(3-hydroxyphenyl)acetamide, 2-(4-amino-4-hydroxyphenyl)-N-(4-methoxyphenyl)acetamide, 2-(4-amino-4-hydroxyphenyl)-N-(3-methoxyphenyl)acetamide, 2-(4-amino-4-hydroxyphenyl)-N-(2,4-dimethoxyphenyl)acetamide, 2-(4-amino-4-hydroxyphenyl)-N-(2-pyridinyl)acetamide, 2-(4-amino-4-hydroxyphenyl)-N-(3-pyridinyl)acetamide, and physiologically compatible water-soluble salts thereof.

5. An agent for the oxidative coloring of keratin fibers based on a developer-substance-coupler-substance combination, wherein said agent comprises at least one o-aminophenol derivative of Formula (I) according to claim 1.

6. An agent according to claim 5, wherein the agent comprises the o-aminophenol derivative of Formula (I) in an amount of from about 0.005 to about 10% by weight of the total weight of the agent.

7. An agent according to claim 5, wherein the agent exhibits a pH value of from about 6.5 to about 11.5.

8. An agent according to claim 5, wherein the agent further comprises at least one dye selected from the group consisting of developer substances, coupler substances, direct-penetrating dyes, and other color components.

9. An agent according to claim 5, wherein the agent is a hair colorant.
